(19) United States
(12) Patent Application Publication
(10) Pub. No.: US 2003/0191509 A1
(43) Pub. Date: Oct. 9, 2003

(54) Method and apparatus for providing custom configured medical devices

(76) Inventors: Timothy M. Flynn, Norton, MA (US); Christina Easterbrooks, Plainville, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/183,065

(22) Filed: Jun. 26, 2002

Related U.S. Application Data
(60) Provisional application No. 60/370,670, filed on Apr. 8, 2002.

(51) Int. Cl. A61N 1/08

(52) U.S. Cl. 607/60

(57) ABSTRACT

A system and method for configuring custom medical devices is provided. The system and method can be used over a communications network, and/or can be provided to users as an application, such as a software program. In general, the system includes a database of medical device components, configuration options available for each component, and rules governing the interdependencies of the components and component options. The system allows a user to select from device components and options for each component to configure a custom built medical device.

### CROSS REFERENCE TO RELATED APPLICATIONS

This application claims priority from U.S. Provisional Patent Application Serial No. 60/370,670, filed on Apr. 8, 2002, entitled “Method and Apparatus for Providing Custom Configured Medical Devices,” which is expressly incorporated by reference herein.

### FIELD OF THE INVENTION

The present invention relates generally to a medical device configurator, and more particularly to methods and systems for electronically configuring custom medical devices.

### BACKGROUND OF THE INVENTION

Medical devices are often purchased based on custom needs and configurations specified by the buyer. Currently, most buyers interact with a medical device professional or sales representative who can assist in meeting their needs. The information exchange generally takes the form of a telephone conversation, sketches, and/or references to standard product offerings. The buyer describes their needs to the medical device professional and relies on that professional's knowledge of the product options and configurations to obtain and/or design the desired medical device. The information provided is often only as reliable as the representative's personal knowledge and experience. As the complexity of some medical devices grows with the number of available options, it becomes more difficult for the medical professional to understand all available options and their interdependencies.

Accordingly, there is currently a need in the medical device industry for an improved method for purchasing custom configured medical devices.

### SUMMARY OF THE INVENTION

The present invention is directed to systems and methods for configuring custom medical devices. The systems and methods can be used over a communications network, and can additionally or alternatively be provided to users as an application, such as a software program. The application can be stored on a computer readable medium, or it can be downloaded via the internet.

In one embodiment, a method for configuring a custom medical device is provided. The method includes the step of providing a medical device database having information about a plurality of medical device components, and at least one configuration option available for each component. The information is presentable on a user-interface. The method further includes the steps of enabling a user to access the database and view the information on a user-interface, enabling the user to input a selection of at least one medical device component from the medical device database, and enabling the user to input a selection of at least one configuration option for each of the at least one medical device components selected. The method can further include the step of configuring an electronic representation of a custom medical device based on the user-selected components and configuration options. In a further embodiment, an electronic image of the custom medical device can be displayed on the user interface. The user can be enabled to input data to change previously inputted selections, and the electronic image of the custom medical device can be updated each time the user inputs data to select from at least one of a component and/or a configuration option.

In another embodiment, a measuring template having a plurality of indicia disposed thereon can be displayed in proximity to the displayed image of the custom medical device. The measuring template can include a movable, sliding scale for allowing a user to measure the dimensions of the custom medical device.

In other aspects, the database can be provided with configuration rules governing the interdependencies of the components and options. Each time a user inputs a selection that violates a configuration rule, the user is preferably notified of the violation. The user can optionally be provided with options for resolving the configuration rule violation.

In further aspects of the invention, the user can save the electronic representation of the custom medical device. Alternatively, or in addition, the user can store the user-inputted selections in a virtual shopping cart, and can optionally place an order for the custom medical device. The “shopping cart” is a tool that allows users to save one or more medical devices of interest to the user. The one or more devices placed in the shopping cart may be stored (preferably temporarily) on the user's computer hard drive for subsequent, local use. Alternatively, the items in the shopping cart are stored in the user's personal file on the system. Thus, each time the user connects to the system, the user can view the items in the shopping cart. Once a user elects to purchase the one or more devices, the user can connect to the system to place an order for the desired items in his/her shopping cart. Any items purchased by the user are subsequently removed from the shopping cart. However, users can view prior purchases (which are stored in the user's personal file) to place subsequent orders for items previously purchased. Users may add or remove items from the shopping cart at anytime. In addition, users can reconfigure devices in their shopping cart at any time and save the reconfigured device as a new configuration.

In an exemplary embodiment, a database having information about at least one pre-built medical device is provided and can be presentable on a user-interface. The information about each of the at least one pre-built medical devices preferably includes pre-selected components and pre-selected configuration options for each of the pre-selected components. The user can be notified when the user-selected components and configuration options are the same as the pre-selected components and pre-selected configuration options of one of the at least one pre-built medical device.

In another embodiment, an electronic configuration system is provided for configuring a custom-designed medical device. The device is electronically viewable on a user-interface of a computer system. The system generally includes a database having information about a plurality of medical device components each having a plurality of user-selectable options, and a user interface that enables the user to input data to select one or more medical device components, and to input data to select options for each component. In use, the system configures an electronic representation of a custom medical device based on the user-selected components and options.

In a further embodiment, the system can include a database having a plurality of configuration rules defining the interdependencies for each of the plurality of medical device components and user-selectable options. The system is preferably adapted to notify the user when a configuration rule is violated, and to provide alternative user-selectable options that are in compliance with the configuration rules. In another embodiment, the system includes a database of images corresponding to each medical device component. The images are viewable through the user-interface. The system can also or alternatively include a database of user-created, custom configured medical devices. The system can also include a personal file for storing user information, custom configured medial devices, order history, etc. The user can access their personal file at any time to place an order, review their order history and status, and to reconfigure a custom configured medical device.

The present invention also provides an interactive, computer-implemented system for configuring an electronic representation of a medical device. The system includes a database having information about a plurality of medical device components and at least one configuration option available for each component, an interface for enabling a user to input data to select at least one medical device component from the medical device database, and to select at least one configuration option for each of the at least one medical device components selected, and a database engine which utilizes the user-inputted data to generate an electronic representation of a custom medical device based on the user-selected components and configuration options.

### BRIEF DESCRIPTION OF THE DRAWINGS

The invention will be more fully understood from the following detailed description taken in conjunction with the accompanying drawings, in which like reference numerals designate like parts throughout the various figures, and wherein:

FIG. 1 is diagram of the general system of the present invention;

FIG. 2 is a diagram illustrating the general method for configuring a medical device according to one embodiment of the present invention;

FIG. 3 illustrates an exemplary embodiment of a medical device configurator system;

FIG. 4 illustrates the step of configuring a custom medical device according to one embodiment of the present invention;

FIG. 5 illustrates the step of selecting configuration options available for one component of a medical device;

FIG. 6 illustrates a method for notifying a user that a conflict between components and/or options exists, and for providing the user with options for resolving the conflict;

FIG. 7 illustrates the step of notifying the user when a custom configured medical device is identical to a pre-built medical device available in a product catalog; and

FIG. 8 illustrates the step of saving a custom medical device configuration according to another embodiment of the present invention.

### DETAILED DESCRIPTION OF THE INVENTION

The present invention is directed to systems and methods for configuring a medical device. The systems and methods can be used over a communications network, and/or can be provided to users as an application, such as a software program, that can be stored on a computer readable medium or downloaded via the internet. In general, the system of the present invention includes a database having information about medical device components, configuration options available for each component, and optionally configuration rules governing the interdependencies of the components and component options. The system allows a user to input data to select device components and options for each component, and configures an electronic representation of a custom medical device based on the user-selected components and options.

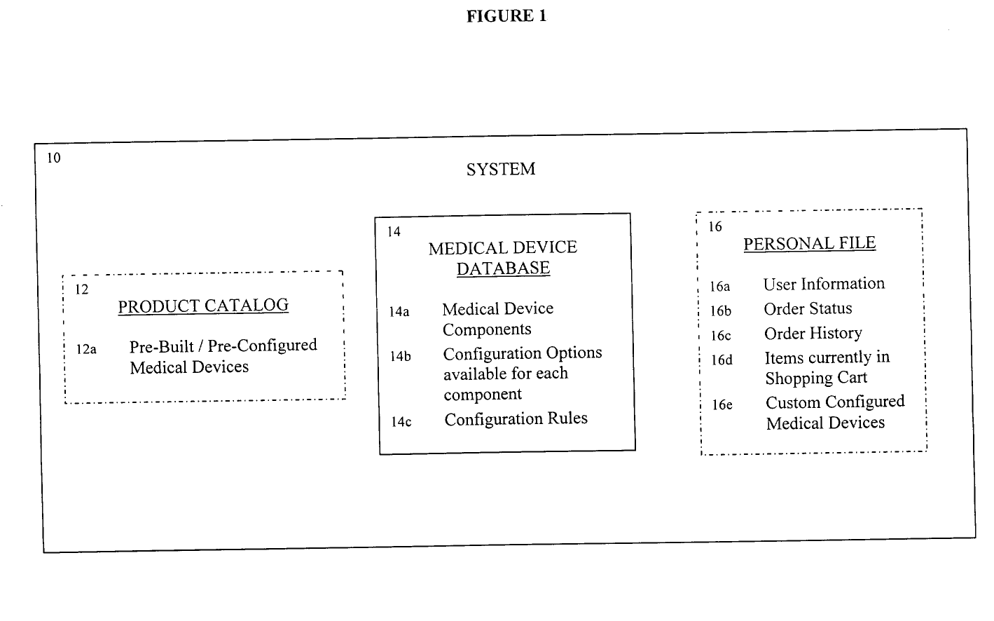

FIG. 1 illustrates an exemplary embodiment of the system 10 of the present invention having a medical device database 14 and, optionally, a product catalog 12 and a personal file 16. The user can access the medical device database 14 to select the components 14a necessary to configure a particular medical device, and to select from available configuration options 14b for each component.

The medical device database 14 contains information about medical device components 14a, and information about configuration options 14b available for each component 14a. The information about the components and configuration options can vary, but preferably the information about each component includes the configuration options for that component, the component features, and other information such as the dimensions, parts, mating elements, function, etc. The information can also optionally include, or be effective to yield, a virtual representation, e.g., a graphic image, of each component. The graphical representation can vary depending on the particular configuration option being applied to the component. The medical device database 14 can also include a set of configuration rules 14c which govern the interdependencies of the components 14a and options 14b available for each component 14a. While FIG. 1 illustrates the configuration rules 14 being stored in the medical device database 14, a person having ordinary skill in the art will appreciate that the configuration rules can be stored in a separate database that is part of the system.

The product catalog 12 is preferably maintained by the system 10 or host site and includes information about a variety of pre-built/pre-configured medical devices 12a that are available for purchase and/or reconfiguration. Each medical device 12a stored in the product catalog 12 includes pre-selected components and pre-selected options for each component. The information can be input into the database by a service provider using a database management tool known in the art. The particular information about each device can be similar to the information stored in the medical device database 14, but preferably includes product codes or similar information corresponding to each pre-built device.

The system 10 can also optionally include a personal file 16 for storing personal date such as, for example, user information 16a, order status 16b, order history 16c, items currently in the user's shopping cart 16d, and custom configured medical devices 16e. While these features are stored in the user's personal file 16, either on the user's hard drive and/or on the system, the features can be integrated with the system 10 such that, for example, the custom configured medical devices 16e can be accessed through the medical device database 14.

A person having ordinary skill in the art will appreciate that the databases of the system can be accessed and represented on a user interface to allow a user to interactively view the information and input data in response to the information presented to the user. The term “interface” as used herein is intended to have a relatively broad definition and can encompass any equipment and/or method for enabling a user to access the system. The interface of the system is preferably controlled by and built into the system.

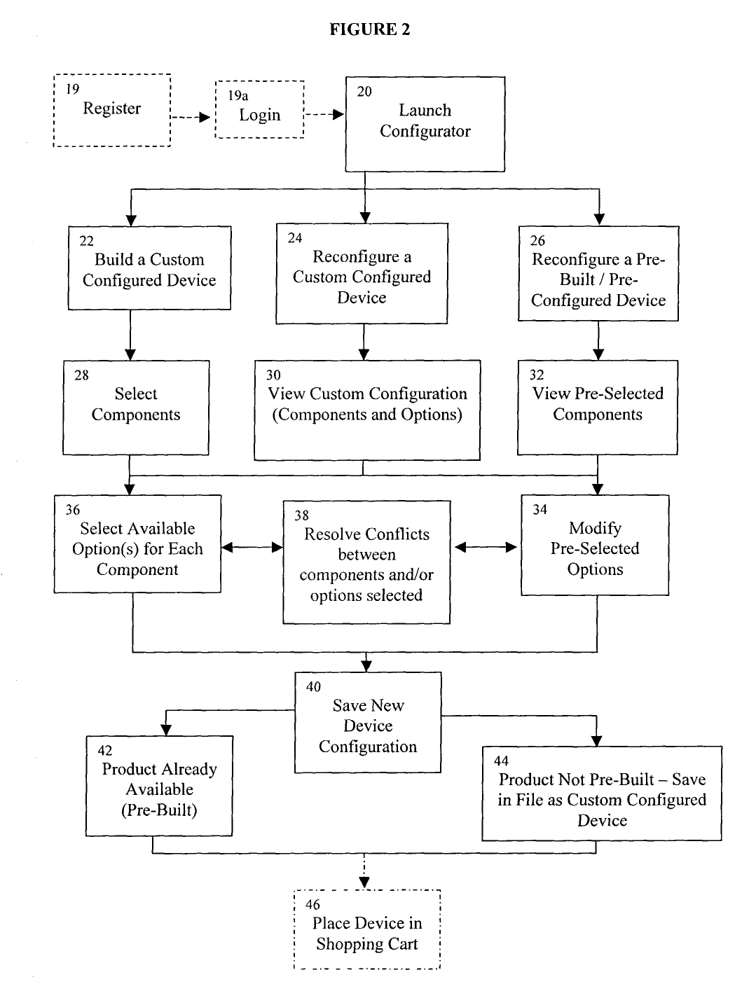

The general method of the present invention is illustrated in FIG. 2. As shown, the user enters the system 10 or host site, preferably via a communications network. The system can optionally include a registration process 19 which requires the user to provide certain user information, and to setup a user login name and a user password. The information submitted in the registration process 19 can be used to configure a personal file 16 for that user. Each time the user logs in 19a to the system, the user's personal file 16 can be retrieved and integrated with the system 10. Once the user is logged into the system 10, the user preferably begins by launching the configurator 20. The user can select from three options: (1) build a custom configured device 22, (2) reconfigure a custom configured device 24 by selecting a custom configured device 14e from the user's personal file 16, or from some other location on the system or on a computer readable medium, or (3) reconfigure a pre-built device 26 by selecting a pre-built device 12a from the product catalog 12. The second option, which allows a user to reconfigure a custom configured device 24, may require the user to have previously accessed the system and to have a custom configured device 14e stored in their personal file 16.

If a user chooses to build a custom configured device 22, the user can begin by selecting components 28 from the medical device database 14. The user is preferably required to select a single component from the medical device database, and then to select all options available for that component prior to selecting a second component. The system will preferably notify the user when components and options for components selected by the user conflict with one another, and require that the user resolve conflicts between the components and/or options selected 38. In an exemplary embodiment, the user is provided with options for resolving the conflict. Once all conflicts are resolved, and the medical device is configured to meet the user's needs, the user can save the device as a new configuration 40. The newly configured device can be stored in the user's personal file 16, and/or can be stored at some other location on the system or on a computer readable medium.

The system can optionally include a method for notifying a user when a custom configured device is identical, e.g., has the same components and component options, to a pre-built device 12a stored in the product catalog 12. When a user chooses to save a custom configured device 40, the system preferably searches through the product catalog 12 for identical pre-built devices and, if an identical device is found, the system notifies the user that the product is already available as a pre-built device 42. The user can be given the option to place the pre-built device in their shopping cart 46. Alternatively, when a custom configured device is not available as a pre-built device, the user can save the custom device as a new configuration 40, as previously described.

In another embodiment, the user can launch the configurator 20 and choose to configure a pre-built device 12a stored in the product catalog 12. The pre-built device is displayed to the user 32 with all components and options pre-selected. The user then has the option to modify the pre-selected components 34, and to modify the pre-selected options for each component 34. If the user modifies the pre-built device, the modified device can be saved as a new custom configured device 40 and stored in the user's personal file 16. As previously stated, if the modified custom configured device, e.g., the new custom configured device 40, is available as a pre-built device, the system will notify the user 42.

Once the user has stored custom configured devices 16e in their personal file 16, the user can login 19a to the system at any time and access their saved custom configured devices 16e. The user can then reconfigured the custom configured device 24 and save the modifications either as a replacement to the previously saved configuration, or as a new configuration.

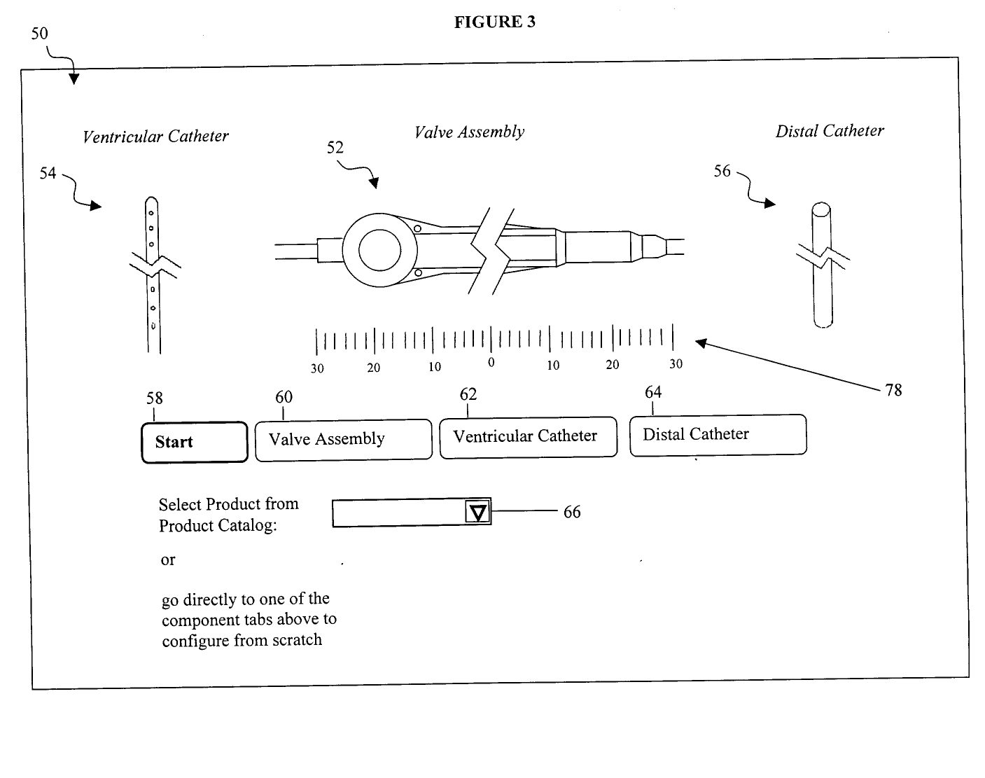

FIGS. 3-8 illustrate an exemplary embodiment, by way of non-limiting example, of a user interface 50 of a medical device configurator system 10 that is designed for configuring a shunt. As shown in FIG. 3, the shunt generally consists of three components: a valve assembly 52, a ventricular catheter 54, and a distal catheter 56. The user beings the configurator by selecting the start button 58 to launch the configurator (FIG. 2, step 20). The user can then build a custom configured device from scratch (FIG. 2, step 22) by selecting one of the component tabs 60, 62, 64, retrieving a previously configured shunt from the user's personal file or some other location (FIG. 2, step 24), or reconfiguring a pre-built shunt selected from the product catalog 12 (FIG. 2, step 26).

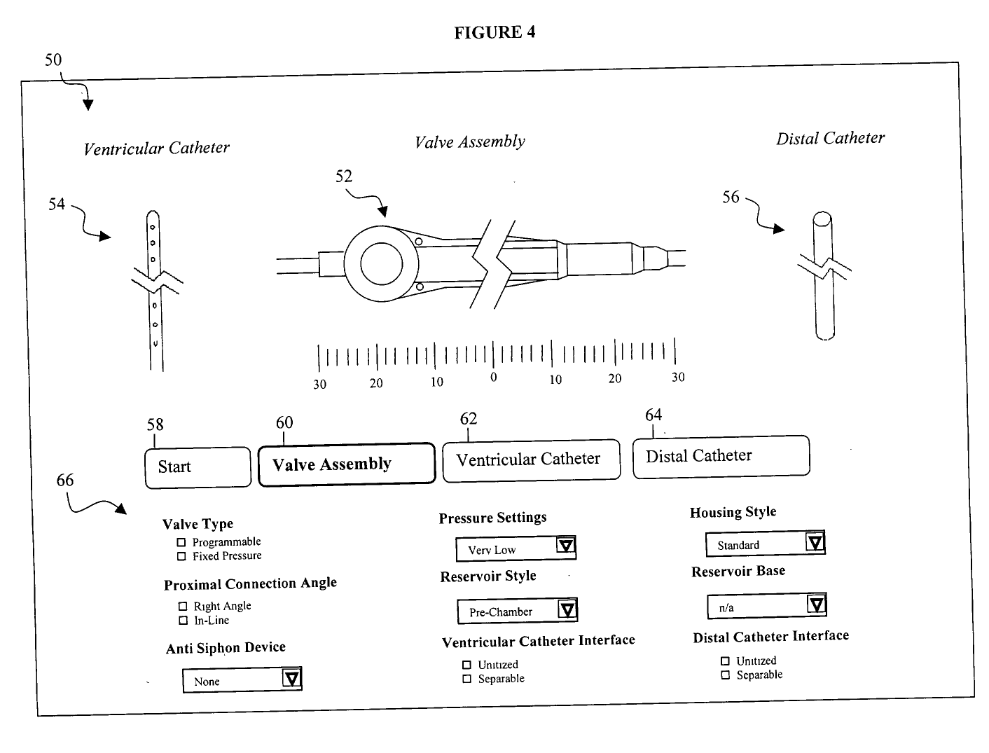

FIG. 4 illustrates the method step of building a custom configured device from scratch (FIG. 2, step 22). The user begins by selecting one of the component tabs 60, 62, 64 to display the configuration options available for that component. As shown in FIG. 4, the user has selected the valve assembly tab 60 to display the configuration options 66 available for the valve assembly 52. The valve assembly 52 includes, for example, the following configuration options: valve type, proximal connection angle, anti siphon device, pressure setting, reservoir style, ventricular catheter interface, housing style, reservoir base, and distal catheter interface. The user then selected the desired setting for each configuration option 66. In a preferred embodiment, the image of the shunt is updated each time the user selects a configuration option. This step is repeated for each of the remaining components of the medical device, e.g., the ventricular catheter and the distal catheter.

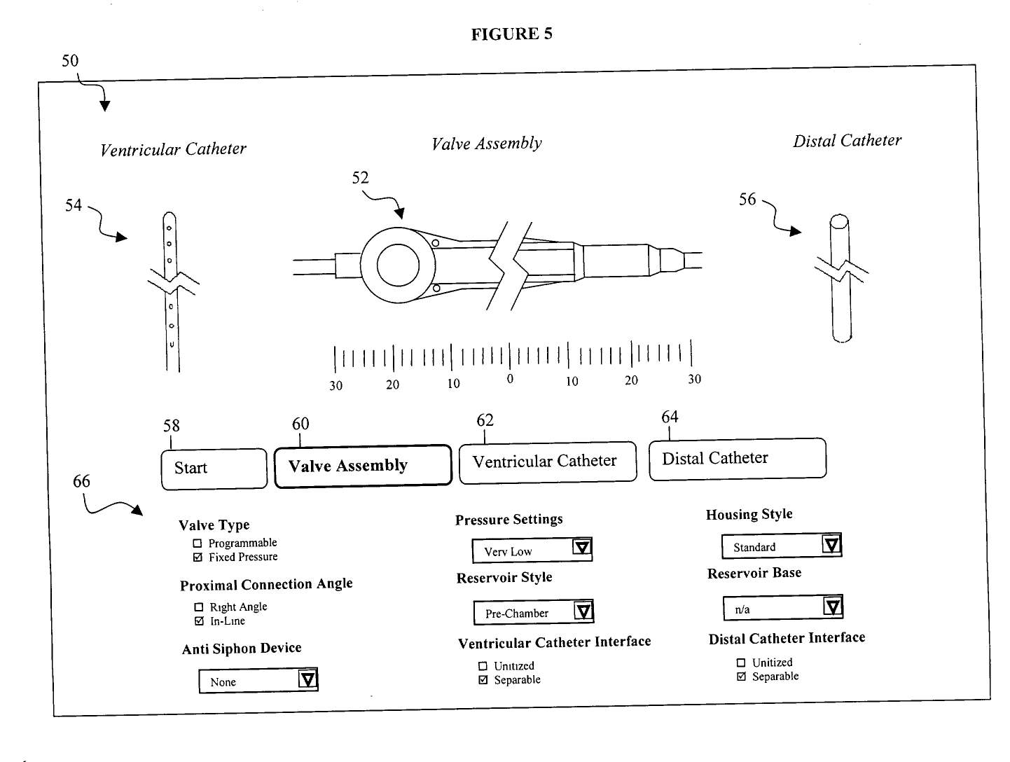

FIG. 5 illustrates the method step of reconfiguring a pre-built shunt (FIG. 2, step 26). The user begins by selecting a pre-built shunt from the product catalog 12, which can be accessed by using the drop-down menu 66, shown in FIG. 3, or alternatively can be displayed on a separate user interface or via other means known in the art. Once the user has selected a pre-built shunt from the product catalog 12, an image of the shunt is displayed. The user can then view the pre-selected options for each component, as shown in FIG. 5, by selecting one of the component tabs 60, 62, 64. The user can optionally change the pre-selected options to reconfigure the shunt.

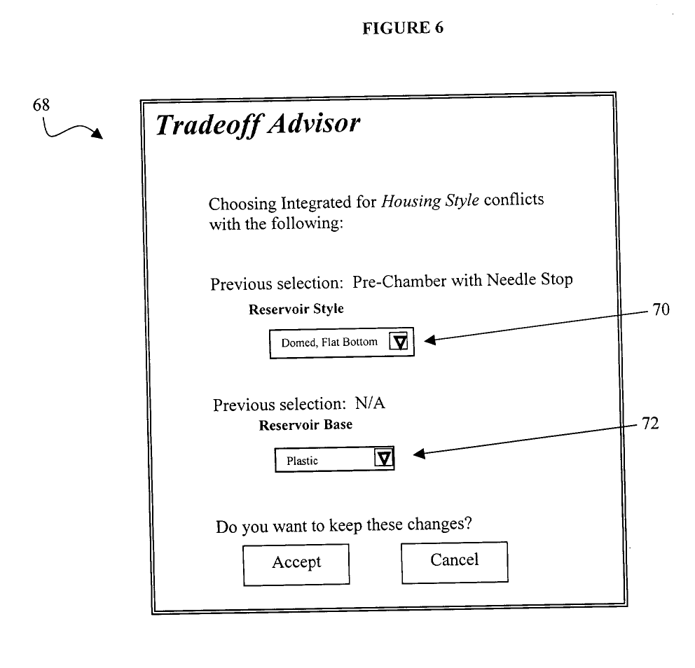

FIG. 6 illustrates another embodiment of the present invention wherein a user is notified if a configuration rule 14c is violated (FIG. 2, step 38). Assume, for example, that the user has selected an integrated housing style, as well as a domed, flat bottom reservoir style and a plastic reservoir base for the valve assembly. Moreover, assume that a configuration rule exists in which an integrated housing style cannot be combined with a flat bottom reservoir or a plastic reservoir base. As shown in FIG. 6, a notification 68 will be displayed for the user to resolve the conflict, and to provide the user with alternative options for resolving the conflict. The user can then either select a different reservoir style or a different reservoir base to resolve the configuration rule violation. A drop-down menu 70, 72 containing alternative options can be provided for each configuration option. Once the user has selected an alternative option, the user can then save the changes and return to the configurator. In the event that the user's selected changes violate another configuration rule, the user can either be prevented from saving the changes, or can be notified of the new configuration rule violation.

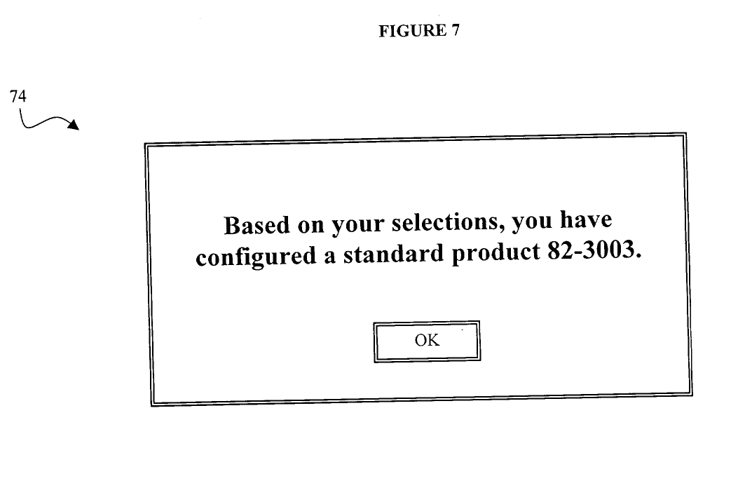

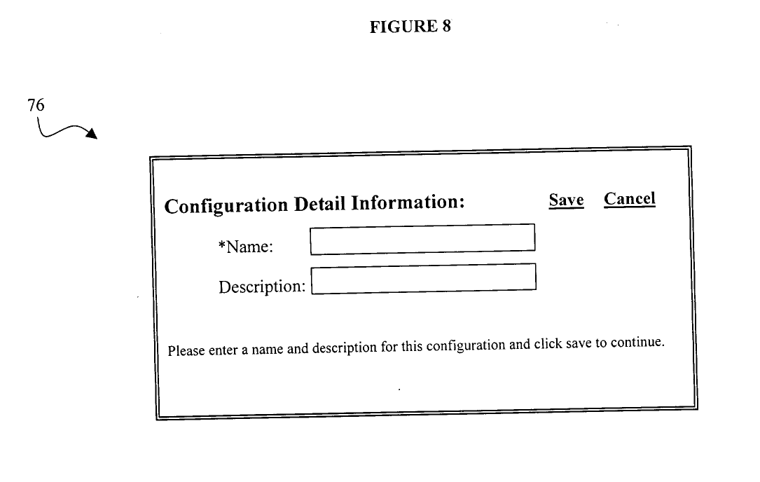

Once the user has completed the configuration, the user can save the custom built shunt configuration (FIG. 2, step 40). However, prior to allowing the user to save the custom configured shunt, the system 10 can optionally search the product catalog 12 for pre-configured shunts having an identical configuration as the custom shunt. If a matching product is found, the system will display a notification 74 to the user, and optionally provide the product name and/or code to the user, as shown in FIG. 7. If a matching pre-built product is not found, the system will prompt the user, via a notification 76, to input a profile for the custom shunt configuration, as shown in FIG. 8. The custom shunt can be saved in the user's personal file 16, somewhere else on the system 10, or on a computer readable medium.

In another embodiment of the present invention, the user interface can include a measuring template, e.g., a ruler, having a movable, sliding scale for allowing a user to measure the dimensions of the medical device being displayed on the user interface. As shown in FIG. 3, the measuring template 78 is formed from a plurality of indicia and a numbering system that is effective to indicate the dimensions of the device. The measuring template 78 is preferably slidably movable in a horizontal direction with respect to the displayed image of the medical device being configured. Alternatively, the device can be slidably movable with respect to the measuring template 78.

The system can also include other features such as, for example, a shopping cart, for saving and optionally purchasing selected medical devices. The “shopping cart” is a tool that allows users to save one or more medical devices of interest to the user. The medical devices can be pre-built devices or custom configured devices. The one or more devices placed in the shopping cart may be stored (preferably temporarily) on the user's computer hard drive for subsequent, local use. Alternatively, the items in the shopping cart are stored in the user's personal file 16 on the system 10. Thus, each time the user connects to the system 10, the user can view the items in the shopping cart. Once a user elects to purchase the one or more devices, the user can connect to the system 10 to place an order for the desired items in his/her shopping cart. Any items purchased by the user are preferably subsequently removed from the shopping cart. However, users can view prior purchases 16c (which are stored in the user's personal file 16) to place subsequent orders for items previously purchased. Users may add or remove items from the shopping cart at anytime. In addition, users can re-configure devices in their shopping cart at any time and save the re-configured device as a new configuration.

The features and other details of the invention will now be more particularly described and pointed out in the claims. It will be understood that the particular embodiments of the invention are shown by way of illustration and not as limitations of the invention. Those having ordinary skill in the art will know, or be able to ascertain, using no more than routine experimentation, many equivalents to the specific embodiments of the invention described herein. These and all other equivalents are intended to be encompassed by the following claims. All publications and references cited herein including those in the background section are expressly incorporated herein by reference in their entirety.

What is claimed is:

1. A method for configuring a custom medical device, comprising:
providing a medical device database having information about a plurality of medical device components and at least one configuration option available for each component, the information being presentable on a user-interface;
enabling a user to access the database and view the information on a user-interface;
enabling the user to input a selection of at least one medical device component from the medical device database; and
enabling the user to input a selection of at least one configuration option for each of the at least one medical device components selected;
configuring an electronic representation of a custom medical device based on the user-selected components and configuration options.

2. The method of claim 1, wherein the medical device database is accessible via a communications network.

3. The method of claim 1, further comprising enabling the user to input data to change previously inputted selections.

4. The method of claim 3, further comprising displaying an updated electronic image of the custom medical device each time the user inputs data to select from at least one of a component and a configuration option.

5. The method of claim 1, further comprising displaying an electronic image of the custom medical device on the user-interface.

6. The method of claim 5, further comprising displaying a measuring template in proximity to the displayed image of the custom medical device, the measuring template having a plurality of indicia disposed thereon.

7. The method of claim 6, wherein the measuring template includes a movable, sliding scale for allowing a user to measure the dimensions on the custom medical device.

8. The method of claim 1, further comprising providing the database with configuration rules governing the interdependencies of the components and options.

9. The method of claim 8, further comprising notifying the user when a user inputs a selection that violates a configuration rule.

10. The method of claim 9, further comprising providing the user with options for resolving the configuration rule violation.

11. The method of claim 1, further comprising enabling the user to save the electronic representation of the custom medical device.

12. The method of claim 1, further comprising enabling the user to store the user-inputted selections in a virtual shopping cart.

13. The method of claim 1, further comprising enabling the user to electronically place an order for the custom medical device.

14. The method of claim 1, further comprising providing a pre-built medical device database having information about at least one pre-built medical device, the information being presentable on a user interface.

15. The method of claim 14, wherein the information about each of the at least one pre-built medical devices includes pre-selected components and pre-selected configuration options for each pre-selected component.

16. The method of claim 15, further comprising notifying the user when the user-selected components and configuration options are the same as the pre-selected components and preselected configuration options of one of the at least one pre-built medical devices.

17. The method of claim 14, wherein the pre-built medical device database is accessible via a communications network.

18. The method of claim 1, wherein the information yields a visual representation of the plurality of medical device components.

19. An electronic configuration system for configuring a custom-designed medical device, the device being electronically viewable on a user-interface of a computer system, the system comprising:
a database having information about a plurality of medical device components, each medical device component having a plurality of user-selectable options; and
a user interface that enables the user to input data to select one or more medical device components, and to input data to select options for each component;
wherein the system configures an electronic representation of a custom medical device based on the user-selected components and options.

20. The system of claim 19, further comprising a database having a plurality of configuration rules defining the interdependencies for each of the plurality of medical device components and user-selectable options.

21. The system of claim 20, wherein the system is adapted to notify the user when a configuration rule is violated, and to provide alternative user-selectable options that are in compliance with the violated configuration rule.

22. The system of claim 19, further comprising a database of images corresponding to each medical device component, the images being viewable on the user-interface.

23. The system of claim 19, further comprising a database of user-created, custom configured medical devices.

24. The system of claim 19, wherein the information yields a visual representation of the plurality of medical device components.

25. An interactive, computer-implemented system for configuring an electronic representation of a medical device, comprising:
a database having information about a plurality of medical device components and at least one configuration option available for each component;
an interface for enabling a user to input data to select at least one medical device component from the medical device database, and to select at least one configuration option for each of the at least one medical device components selected; and
a database engine which utilizes the user-inputted data to generate an electronic representation of a custom medical device based on the user-selected components and configuration options.
